(19) United States
(12) Patent Application Publication
(10) Pub. No.: US 2025/0000460 A1
(43) Pub. Date: Jan. 2, 2025

(54) SYSTEM AND METHOD FOR DETERMINING FALL RISK

(71) Applicant: JASPER MEDTECH PTY LTD, Sydney, New South Wales (AU)

(72) Inventor: Ralph MOBBS, Sydney, New South Wales (AU)

(21) Appl. No.: 18/578,714

(22) PCT Filed: Jul. 12, 2022
(86) PCT No.: PCT/AU2022/050728

§ 371 Date: Jan. 11, 2024

(30) Foreign Application Priority Data
Jul. 12, 2021 (AU) 2021902126
Aug. 23, 2021 (AU) 2021106605

(51) Int. Cl. A61B 5/00; A61B 5/11; G16H 50/30

(52) U.S. Cl. CPC A61B 5/7275; A61B 5/112; G16H 50/30; A61B 2562/0219; A61B 2562/0223

(57) ABSTRACT

There is provided a system and method for determining a fall risk of a subject a gait stability score for the subject from at least two gait metrics or a walking orientation randomness metric (WORM) score. If one or more of the scores falls outside a predetermined range or above a predetermined threshold the subject is at risk of falling.

### TECHNICAL FIELD

The present invention relates to a system and method for determining a fall risk for a subject utilizing a combination of Spatio-Temporal gait metrics and/or a stability metric; the Walking Orientation Randomness Metric (WORM).

### CROSS REFERENCE TO RELATED APPLICATIONS

This application claims priority to Australian provisional application number 2021902126, filed 12 Jul. 2021, and Australian innovation patent number 2021106605, filed 23 Aug. 2021. The disclosure of both applications is incorporated by reference in their entireties.

### BACKGROUND

The observation and management of a subject after a medical or surgical intervention is challenging and resource intensive. Monitoring patients, or population based general health metrics, is important to avoid preventable complications including falls in risk individuals, and general health deterioration.

At present, monitoring is predominantly performed by healthcare staff, or using questionnaires, or with the assistance of sensors/sensor systems which allow single and multiple parameter monitoring to occur in the hospital, healthcare facility or outside these controlled environments (home or other) in less constrained environments where the subject will engage in normal activities.

Monitoring gait stability is possible in a subject's normal environment where they are carrying out normal activities with little or no constraint. This can involve the monitoring of various physiological parameters during normal daily activities. For example, during monitoring, a subject may be walking, exercising, engaging in a rehabilitation program or working at either sedentary or active tasks.

Devices to monitor posture or gait stability are known. These devices typically use at least one accelerometer to monitor posture, gait or both in real time. In some instances a wearable monitoring device is attached to a subject, for example by a belt and is aligned to the subjects midsagittal plane. Other technology uses a pressure-sensing mat, or “walkway,” to measure the relative arrangements of the footfalls as a person walks across the mat, in conjunction with software to process the footfalls to derive certain spatiotemporal gait parameters, such as, e.g., stride length. While this system constitutes the current “gold standard” for gait measurements, it fails to capture, by its nature, the three-dimensional (3D) movements associated with walking, and is impractical and too costly to use in a healthcare setting. An alternative approach utilizes marker-based motion capture in conjunction with a biomechanical model to derive kinematic parameters. This system is also impractical in a healthcare setting. As a result, gait stability monitoring is not used to assess the fall risk of subjects in normal day to day living, or post medical intervention.

Conventional methods of falls prediction typically use gait velocity and step length as predictors of falls, and advents such as turning kinetics are in an immature stage of research. In addition the bulk of studies examine community-dwelling elderly, with few investigating neurogenic gait alterations and none are specific for spinal neurogenic gait alterations.

Accordingly, there is a need for improved systems and methods to provide robust and reliable extraction of gait metrics to provide useful clinical information such as the risk of a fall, the subject's suitability or need for a walking aid, or suitability for discharge from hospital or other healthcare facility.

### SUMMARY

In a first aspect there is provided a system for determining a fall risk of a subject, the system comprising:
a) an accelerometer configured to output signals indicative of movement of the subject along one or any combination of an x-axis, a y-axis, and a z-axis
b) a magnetometer configured to output signals indicative of variations in position of the subject in a space defined by the x-axis, the y-axis, and the z-axis; and
c) a gyroscope configured to output signals indicative of angular velocity of the subject around one or any combination of the x-axis, the y-axis, and the z-axis;
d) a processor configured to receive the output and analyse the signals to determine for the subject one or any combination of gait metrics:
   i. stride time;
   ii. stride time variability;
   iii. stride cadence;
   iv. step time asymmetry;
   v. stride length;
   vi. stride length variability;
   vii. stride length asymmetry;
   viii. gait velocity;
   ix. gait speed variability; and
   x. walking orientation randomness metric (WORM)
wherein the x-axis is a horizontal axis to the ground directed forward of the subject's body; the y-axis being a horizontal axis to the ground directed laterally of the subject's body; and the z-axis is vertical axis to ground.

The accelerometer, magnetometer and gyroscope may be in a sensor unit adapted to be disposed on the subject.

The sensor unit may further comprise the processor.

The processor may be configured to determine a gait stability score from any two, three, four, five, six, seven, eight, or nine of gait metrics i-ix.

The WORM Score may be determined by:
i) calculating the point pₜ at time step t from the orientation of the body with respect the world frame, ^{W}Rₜ^{B}.
ii) obtaining the body orientation, ^{W}R^{B}, from the orientation, ^{W}R̆ₜ^{S}, adjusted by a fixed sensor-to-body rotational offset, ^{B}R₀^{S};
wherein the sensor-to-body rotational offset, ^{B}R₀^{S}, is calculated by assuming an upright pose
iii) calculating point pₜ, being the x and y coordinates of the body z axis with centre at the origin; and
wherein the WORM score is the distance travelled in the transverse plane travel by pₜ.

The body orientation, ^{W}R^{B}, may be calculated from the orientation, ^{W}R̆ₜ^{S}, adjusted by the fixed sensor-to-body rotational offset, ^{B}R₀^{S}, according to equation 15:

$${W}^{} {R}_{t}^{B} = {}^{W} {{R}^{⌣}}_{t}^{S} {( {}^{B} {R}_{0}^{S} )}^{T} \qquad Equation 15$$

The sensor-to-body rotational offset, ^{B}R₀^{S}, may be calculated by assuming an upright pose (^{W}R₀^{B}=I_{3×3}) at t=0 according to equation 16:

$${}^{B} {R}_{0}^{S} = {}^{W} {{R}^{⌣}}_{0}^{S} \qquad Equation 16$$

Point pₜ, may be calculated using equation 17:

$${p}_{t} = [ \begin{matrix}1 & 0 & 0 \\ 0 & 1 & 0\end{matrix} ] {}^{W} {R}_{t}^{B} [ \begin{matrix}0 \\ 0 \\ 1\end{matrix} ] \qquad Equation 17$$

The system of any one of claims 1 to 3, wherein the WORM Score is calculated using Equation 14:

$$WORM = \frac{{\sum }_{i = 1}^{n} Li}{n} \qquad Equation 14$$

where Li is total length of the path taken by a point X relative to a point O in the horizontal plane during a meter i of a walking bout of n meters.

In one embodiment the gait stability score may be determined by summing:
gait velocity and step length;
cadence and step time;
step time asymmetry and step length asymmetry; or
   gait velocity variation, step time variation and step length variation. In a second aspect there is provided a method of determining a fall risk of a subject comprising determining a gait stability score for the subject from at least two of
i. stride time;
ii. stride time variability;
iii. stride cadence;
iv. step time asymmetry;
v. stride length;
vi. stride length variability;
vii. stride length asymmetry;
viii. gait velocity;
ix. gait speed variability; or
wherein fall risk is determined by calculating a walking orientation randomness metric (WORM) score wherein one or both of the gait stability score or WORM score is indicative of a fall risk.

In one embodiment a WORM score of 2.5 or above is indicative of a fall risk.

In one embodiment a WORM score of above a predetermined threshold is indicative of a high fall risk (high fall risk threshold). For example the high fall risk threshold may be 1.2.

In one embodiment a WORM score below a predetermined threshold indicative of a minimal risk of falling (minimal fall risk threshold), for example the minimal fall risk threshold may be 0.2.

In one embodiment a WORM score between a minimal fall risk threshold and a high fall risk threshold is indicative of a low risk of falling, for example the minimal fall risk threshold may 0.2 and the high fall risk threshold may be 1.2.

In one embodiment a WORM score of between a medium fall risk threshold and a high fall risk threshold is indicative of a medium risk of falling, for example the medium fall risk threshold may be 0.6 and the high fall risk threshold may be 1.2.

In another embodiment the gait stability score is the sum of gait velocity and step length and a gait stability score of 1.4 or less is indicative of a fall risk.

In another embodiment the gait stability score is the sum of cadence and step time and a gait stability score of 95 or less is indicative of a fall risk.

In another embodiment the gait stability score is the sum of step time asymmetry and step length asymmetry and a gait stability score of 0.25 or more is indicative of a fall risk.

In another embodiment the gait stability score is the sum of gait velocity variation, step time variation and step length variation and a gait stability score of 0.25 or more is indicative of a fall risk.

### Definitions

As used herein the term ‘WORM’ refers to Walking Orientation Randomness Metric

As used herein the term ‘IMU’ refers to an Inertial Measurement Unit.

The term ‘AP’ refers to Antero-Posterior.

The term ‘ML’ refers to Medio-Lateral.

The term ‘MEMS’ refers to Micro Electro Mechanical Sensors.

Throughout this specification, unless the context clearly requires otherwise, the word ‘comprise’, or variations such as ‘comprises’ or ‘comprising’, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (ie, at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

In order that the present technology may be more clearly understood, preferred embodiments will be described with reference to the following drawings and examples.

### BRIEF DESCRIPTION OF THE DRAWINGS

Embodiments of the systems and methods are described with reference to the following drawings.

FIG. 1 is a schematic diagram showing preferred locations of the wearable sensor device. Locations may be anterior or posterior.

FIG. 2 is a schematic diagram of an embodiment of a system for monitoring a subject after surgery and includes a user interface for a display device to display information obtained from the wearable sensor device. This embodiment is one example of how the technology may be used, and is not limiting.

FIG. 3 is a schematic diagram illustrating how the sensor device determines the WORM score or WORMdist which is calculated from movement around and/or along x, y and z, axes.

FIG. 4 is a summary of data collection, processing, and outputs from the MetaMotionC sensor and data processing for gait analysis used in this study. FIG. 4a. First output is a .html file which documents the vertical acceleration measured by the sensor (y-axis) against time (x-axis) during the walk done by the participant. Green circles represent the initial foot contact with the ground, usually the ‘heel strike’ phase of gait and orange circles represent the final foot contact with the ground, usually the ‘toe-off’ phase of gait. FIG. 4b. The data processing uses the gait cycle events detected in image A. to identify when a gait cycle begins and ends, and thus creates a .csv file with the values of each gait parameter displayed per gait cycle and for the bout overall. WORM=walking orientation randomness metric, MMC=MetaMotionC sensor from Mbient Labs (or any other 9-axis accelerometer), used to measure gait in the present study.

### DESCRIPTION OF EMBODIMENTS

Postural and ambulatory control of balance and stability is an important component of gait and is closely related to falls risk and can be an indicator of neurological and/or musculoskeletal pathologies.

The present invention is directed to systems and methods for determining the risk of a fall from various gait metrics. The technology uses non-invasive systems and methods to assess and assist with falls prediction which in turn informs the suitability or necessity for the use of a walking aid such as a walking stick or walking frame; and the suitability of a patient to be discharged from a hospital or other healthcare facility.

The technology is useful for assessing the level of physical disability in the context of recovery from a surgical or medical intervention, particularly in monitoring the effectiveness of an ambulation protocol which can improve patient outcomes, but is often overlooked by healthcare staff who have competing clinical duties.

Two-thirds of the body mass is located two-thirds of the height above the ground. To move forward during walking, the center of gravity (upper body) is accelerated forwards of the base of support (lower body). With each step, this center of gravity also oscillates laterally along the line of motion.

A fall is an event where a person inadvertently comes to rest on the ground, typically while attempting to ambulate. Patients who fall suffer physical harm with potentially lethal consequences.

Balance is needed to keep the body oriented appropriately while performing voluntary activity such as ambulation, during external perturbations and when the support surface or environment changes. Balance or postural stability requires three distinct processes: (i) sensory organization, in which one or more of the orientational senses (somatosensory, visual and vestibular) are involved and integrated within the CNS; (ii) a motor adjustment process involved with executing coordinated and properly scaled neuromuscular responses; and (iii) the background tone of the muscles, through which changes in balance are affected. In patients that have had a surgical or medical intervention the motor adjustment process may be impaired for a period after the intervention.

Organization of the orientational senses is understood to be an adaptive hierarchical system. There are two main reference frames for the sensory representation of the body posture with respect to space. On a lower level, a weighted combination of orientational inputs directly mediates the activity of postural muscles and mainly controls the horizontal centre of gravity (COG) position. On a higher level, vestibular inputs provide the orientational reference, against which conflicts in support surface and visual orientation are identified and the combination of inputs adapted to the task conditions. For postural stability, the information from the lower level must be coherent with the inertial-gravitational reference of the higher level, and any conflicting orientation inputs must be quickly suppressed in favour of those congruent with the internal reference. Thus, in adults, the sensory organizational process is context specific due to the rapid weighting and re-weighting of sensory inputs to/from the lower level by the higher level adaptive process.

The systems and methods disclosed herein comprise one or more Inertial Measurement Units (IMUs), commonly known as ‘wearable devices’ or ‘wearables’ which contain various microelectromechanical sensors (MEMS) including accelerometers, gyroscopes and magnetometers. IMUs and are an alternative to the existing methods of gait assessment in laboratory based clinical settings. Wearables can accurately measure numerous gait metrics including gait velocity, stride length, cadence, and step count. Accordingly, the systems and methods disclosed herein can be used to monitor a subject's recovery from surgery or other treatment, to monitor the healing process, or to monitor or verify the extent of the subject's activity, or any combination of these purposes.

In some embodiments the systems and devices described herein utilise various gait metrics to determine the Walking Orientation Randomness Metric, or WORM score. The WORM score can be used to quantitatively measure the stability, or ‘wobble’, of a subject during ambulation.

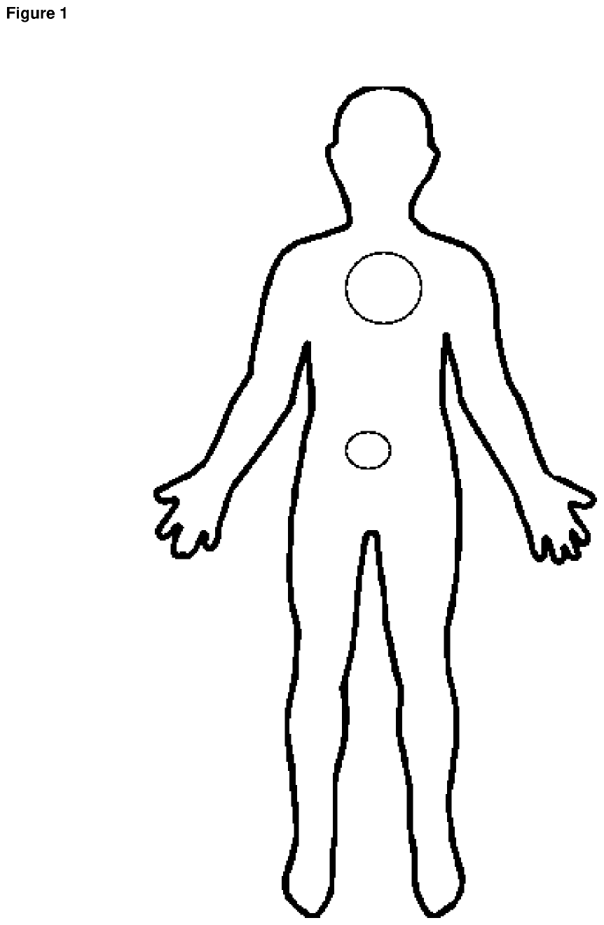

As shown in FIG. 1, the systems and methods use a sensor device placed on a subject's chest, low back, belt line or the like. The sensor may be anterior or posterior. The system includes one or more sensor devices that communicate with a processor that can produce information, based on the sensor readings and data, to facilitate the patient or another user, such as a clinician, doctor, hospital, carer, or other appropriate person, monitor the subject.

The system includes a wearable device with one or more sensors, such as accelerometers. For example, the wearable device may include one or more sensors and may be applied to the skin of a subject. In at least some embodiments, the one or more sensors communicate with a processor. The processor may be in the wearable device or may be remote from it. In some embodiments the sensor device also includes a display. In some embodiments, the processor, the sensors, or both communicate with a display device, such as a mobile phone, tablet, or computer.

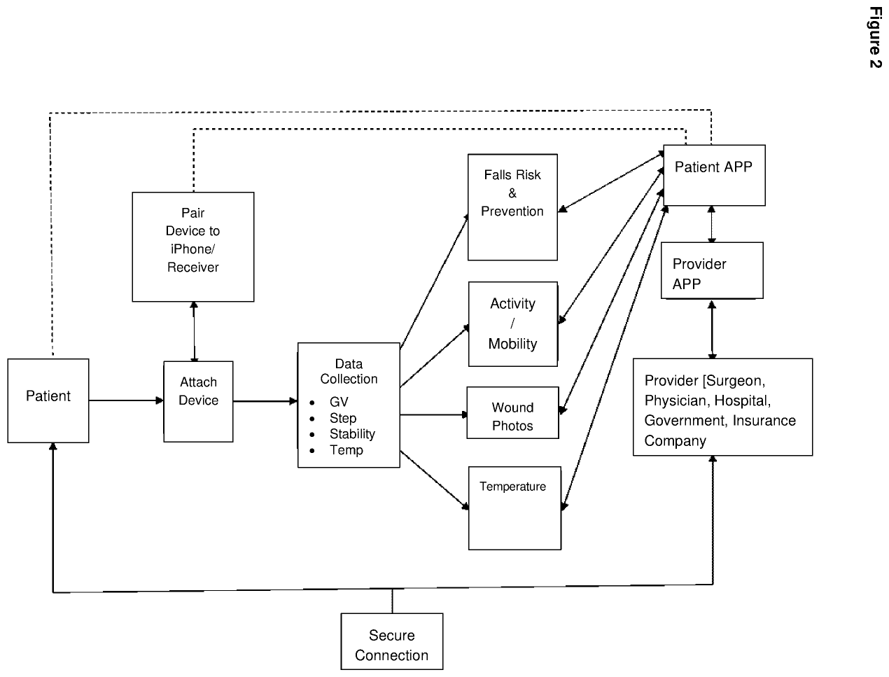

FIG. 2 illustrates one embodiment of a system for monitoring, for example, a patient's mobility and stability post intervention. The system includes a processor, and one or more sensors in a wearable device.

Optionally, the system includes a display device, such as a mobile phone, tablet, or computer that may comprise the processor and may be used to process and/or display information obtained or derived from the sensor device.

In at least some embodiments, the one or more sensors and, preferably, the processor (or multiple processors) are provided in a sensor device that is adapted to be applied to the skin of the patient, carried on an article of clothing or carried on a sling or harness worn by the patient.

The display device can be any suitable device such as a computer (for example, a notebook or laptop computer, a mobile medical station or computer, a server, a mainframe computer, or a desktop computer), mobile devices (for example, a smartphone, smartwatch, or a tablet), or any other suitable device. In some embodiments, the display device can be incorporated into a medical station or system.

In some embodiments the display device is configured to communicate with one or more other devices and can for example alert a subject's clinician, career or other designator person or service. For example if the gait metrics and/or WORM score indicate that the subject is at a high risk of falling an alert may be sent to a carer or clinician. Alternatively if the example if the gait metrics and/or WORM score indicate that the subject is ambulating effectively an alert may be sent to a clinician or an electronic medical record that the subject is ready for discharge.

In one embodiment of the sensor device, the display device, or both have the ability to process data and comprise a memory, a display, and are adapted to receive an input via an input device. In some embodiments these components can be carried by the user (for example if they are part of the sensor device).

The processor is configured to execute instructions provided to the processor. Such instructions can include any of the steps of methods or processes described herein. Any suitable memory can be used for the sensor and display devices. The memory may be any computer-readable storage media such as, nonvolatile, non-transitory, removable, and non-removable computer-readable media implemented in any method or technology for storage of information, such as computer readable instructions, data structures, program modules, or other data.

Communication methods provide another type of computer readable media, e.g. communication media. Communication media typically embodies computer-readable instructions, data structures, program modules, or other data in a modulated data signal such as a carrier wave, data signal, or other transport mechanism and includes any information delivery media. By way of example, communication media includes wired media such as twisted pair, coaxial cable, fiber optics, wave guides, and other wired media and wireless media such as acoustic, RF, infrared, Bluetooth′, near field communication, and other wireless media.

The display can be any suitable display such as a monitor, screen, display, or the like, and can include a printer.

The input device can be, for example, a keyboard, mouse, touch screen, track ball, joystick, voice recognition system, camera, microphone, or any device known in the art to provide input directly or indirectly to a processor . . .

Any suitable type of sensor can be used including, but not limited to, accelerometers, magnetometers, gyroscopes, proximity sensors, infrared sensors, ultrasound sensors, thermistors or other temperature sensors, cameras, piezoelectric or other pressure sensors, sonar sensors, external fluid sensor, skin discoloration sensor, pH sensor, and microphones or any combination thereof.

In at least some embodiments, the system includes at least one, two, three, four, five, six, or more different types of sensors. The system may include at least one, two, three, four, five, six, eight, ten, or more sensors. The sensors may be present in a single sensor device or in multiple sensor devices adapted to be applied to different areas of the subject.

The one or more sensor devices can be used to measure, monitor, or otherwise observe a subjects gait metrics and therefore their physical activity or health; recovery from surgery or other treatment; rehabilitation program, or any combination thereof.

Information sufficient to calculate one or more o the following can be obtained by the sensors: gait velocity, step length, step cadence, step time, step time asymmetry, step length asymmetry, gait velocity variation, step time variation, and step length variation. Other examples of observations or measurements that can be made or interpreted using one or more of the sensors include activity, temperature of skin, pulse or pulse profile or heart rate recovery time after activity, sleep profile or rest duration. The system can observe or measure one or more of these items or any combination of the items.

The sensor device may be adapted to adhere to the skin or otherwise be held adjacent to the skin of the subject. The sensor device typically includes a housing and an adhesive pad to attach the base to the skin of the subject. Alternatively the housing may be adapted to attached to an article of clothing. Within the housing the sensor device comprises one or more sensors, a power source, a communications unit, and optionally a processor.

The housing can be made of any suitable material, such as plastic or silicone, and has sufficient flexibility to fit comfortably to or rest adjacent to the subject's skin. In some embodiments the housing is also resistant to water, sweat, and other fluids. In some embodiments the housing is sufficiently water resistant to allow the patient to shower or bathe with the sensor device.

In some embodiments the sensors, power source, communications unit, and processor are contained within the housing. In some embodiments, a portion of one or more of the sensors, such as a temperature, pulse, or pressure sensor, moisture sensor, or strain gage, may protrude through the housing to allow contact of the sensor or part o the sensor with the skin of the patient.

In some embodiments of the sensor device comprises an accelerometer, a gyroscope and a magnetometer. The accelerometer, gyroscope and magnetometer can be used to measure gait metrics as noted above.

Other suitable sensors include, but are not limited to, a microphone, pulse oximetry sensor, a heart rate monitor, or the like, or any combination thereof. As will be understood, any suitable sensor described above can be included in the sensor unit and any combination of those sensors can be used in the sensor unit.

Power can be provided to the sensors and processor using any suitable power source such as primary cells, coin cell batteries, rechargeable batteries, storage capacitors, other power storage devices, or any combination thereof. In some embodiments, the power is provided by a kinetic energy power to power the components or to or to recharge a battery or other power source coupled to the components. In some embodiments, a wireless power source can be used. In some embodiments the sensor device comprises a charging port for charging the power source. Alternatively or in addition, wireless charging systems and methods can be used.

All of the sensors and the processor may be coupled to the same power source or some of the sensors (or even all of the sensors) and sensor processor may have individual power sources.

In some embodiments, the sensors and processor are continuously active. In other embodiments, the sensors and processor are active intermittently (for example every 0.1, 0.5, 1, 5, 10, 15, or 30 seconds). Optionally, the period may be programmable. In one embodiment the period is altered based on data from one or more of the sensors. In another other embodiment the sensors and processor are activated manually or automatically by the sensor device or display device. In some embodiments the sensors and processor are activated automatically when the sensor device is put into motion.

In some embodiments, each sensor may have different activation schedules (e.g. continuous, intermittent, manual). For example, a temperature sensor may measure temperature periodically, a sensor to measure gait velocity or step asymmetry may be activated automatically when motion is detected.

The processor can be any suitable processor and may include, or be coupled to memory for storing data received from the sensor. The processor can be wired or wirelessly coupled to the sensor. In some embodiments, the processor may include analysis algorithms for analyzing or partially analyzing data received from the sensor. In other embodiments, the processor may be used to receive, store, and transmit data received from the sensors.

The communications unit can be any suitable communications arrangement that can transmit information from the processor or sensors to another device (such as the display device) The communications unit can transmit this information by any suitable wired or wireless technique such as Bluetooth, near field communications, WiFi, infrared, radio frequency, acoustic, optical, or by a wired connection through a data port in the sensor device.

The systems and methods can utilise personal characteristics of the subject to assist in determining one or more gait metrics or WORM score. The personal characteristics can include one or any combination of age, gender, height, weight, level of activity, level of mobility, body mass index (BMI), leg length discrepancy, and surgical procedure. In some embodiments, the gait metrics or WORM score may differ based on the subject's gender, age, or height (or any other personal characteristic or combination of personal characteristics).

In at least some embodiments, the ranges for the different measurements can be modified for age, gender, height, or other personal characteristics, or any combination thereof. An application on the display device may provide information regarding the measurements (for example, lists of the measurements, graphs of the measurements, averages or daily numbers for the measurements or the like or any combination thereof), as well as any of the metrics described above such as the WORM score. The application may allow a user to access to some or all profile details and may permit access to sensor unit set-up and calibration applications or protocols.

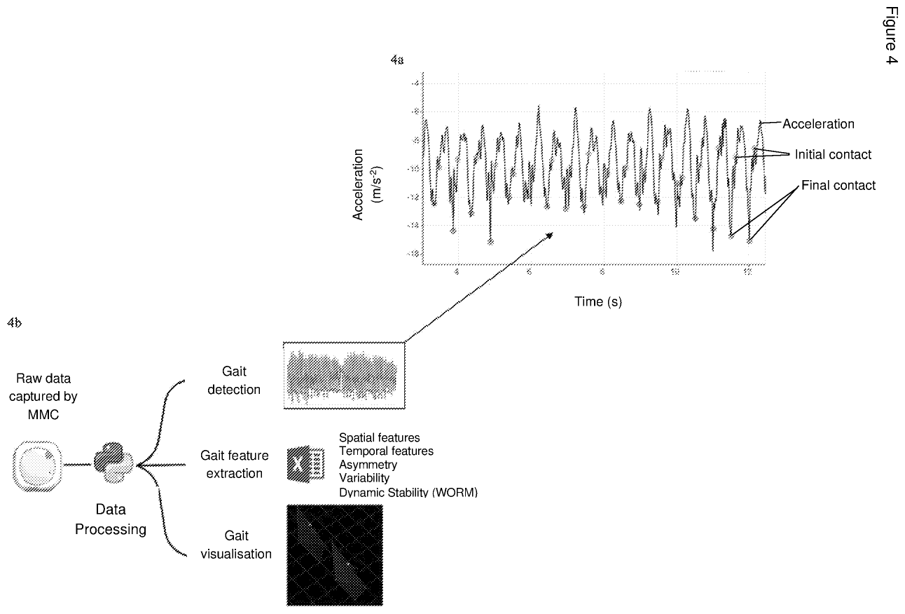

In some embodiments commercially available sensors and software packages such as the MetaMotionC sensor (or any other 9-axis accelerometer) may be used for gait analysis. However, a skilled person will be able to create suitable code for data collection from the sensor (for example a 9-axis accelerometer), data processing, and outputs for gait analysis. The first output from the IMU may be a .html file which documents the vertical acceleration measured by the sensor (y-axis) against time (x-axis) during the walk done by the subject. Green circles represent the initial foot contact with the ground, usually the ‘heel strike’ phase of gait and orange circles represent the final foot contact with the ground, usually the ‘toe-off’ phase of gait. FIG. 4b. The program uses the gait cycle events detected in image a to identify when gait cycles begin and end, and thus creates a .csv file with the values of each gait parameter displayed per gait cycle and for the bout overall, these data can be use to calculate gait metrics (see below). Additionally, a .c3d file is created which can be viewed using any suitable viewer known in the art, for example Mokka (an open source platform), to create a visual recreation of the gait using the accelerometry data. WORM=walking orientation randomness metric.

Wearable sensors can sample data at a range of rates. For example, as exemplified herein the sensor at a rate of 100 hz. However, it is envisaged that sample rates from around 20 Hz to 600 Hz, for example suitable sampling rates may be 20, 30, 40, 50, 60, 70, 80, 90, 100, 125, 150, 175, 200, 225, 250, 275, 300, 325, 350, 375, 400, 425, 450, 475, 500, 525, 550, 575, or 600 Hz. Sampling rates in excess of 600 Hz are also compatible with the methods and systems described herein.

### Gait Metrics

The systems and methods described herein utilise one or more of the following gait metrics. While the gait metrics can be calculated using any known methods, the exemplary methods below assume that n steps were taken over the entire bout, or for the walking orientation randomness metric calculation, that the bout was n meters long.

ST=stride time, STV=stride time variability, SL=stride length, SLV=stride length variability, GV=gait velocity, GSV=gait speed variability, WORM=walking orientation randomness metric.

$$Average ST = \frac{{\sum }_{i = 1}^{n} Time of stride i}{n} \qquad Equation 1$$

$$STV = \frac{\sqrt{{\sum }_{i = 1}^{n} {( Time of stride i - Average ST )}^{2}}}{Average ST \sqrt{n - 1}} \qquad Equation 2$$

$$Average step time = \frac{\sqrt{{\sum }_{i = 1}^{n} Time of stride i}}{2 n} \qquad Equation 3$$

$$Average cadence = \frac{60}{Average step time} \qquad Equation 4$$

$$Step time variability = \qquad Equation 5$$

$$\frac{\sqrt{{\sum }_{i = 1}^{2 n} {( Time of step i - Average step time )}^{2}}}{Average step time \sqrt{2 n - 1}}$$

$$Average step time asymmentry = \qquad Equation 6$$

$$\frac{{\sum }_{i = 1}^{n} Time of step 2 i - Time of step 2 i - 1}{n}$$

$$Average SL = \frac{{\sum }_{i = 1}^{n} Length of stride i}{n} \qquad Equation 7$$

$$SLV = \frac{\sqrt{{\sum }_{i = 1}^{n} {( Time of stride i - Average SL )}^{2}}}{Average SL \sqrt{n - 1}} \qquad Equation 8$$

$$Average step length = \frac{\sqrt{{\sum }_{i = 1}^{2 n} Length of step i}}{2 n} \qquad Equation 9$$

$$Step length variability = \qquad Equation 10$$

$$\frac{\sqrt{{\sum }_{i = 1}^{2 n} {( Length of step i - Average step length )}^{2}}}{Average step length \sqrt{2 n - 1}}$$

$$Average step length asymmetry = \qquad Equation 11$$

$$\frac{{\sum }_{i = 1}^{n} Length of step 2 i - Length of step 2 i - 1}{n}$$

$$Average GV = \qquad Equation 12$$

$$\frac{Total distance}{Total time} = \frac{Average SL}{Average ST} = \frac{{\sum }_{i = 1}^{n} GV during stride i}{n}$$

$$GSV = \frac{\sqrt{{\sum }_{i = 1}^{n} {( GV during stride i - Average GV )}^{2}}}{Average GV \sqrt{n - 1}} \qquad Equation 13$$

### Walking Orientation Randomness Metric (WORM) Score

In one embodiment Let Li be the total length of the path taken by point X relative to point O in the horizontal plane during meter i of a walking bout of n meters.

$$WORM = \frac{{\sum }_{i = 1}^{n} Li}{n} \qquad Equation 14$$

During walking, the summative motions of individual joint segments accelerates the trunk forwards of the base of support. With each step, the trunk also oscillates laterally along a line of motion situated at the medial borders of either feet.

To reflect the aforementioned ‘inverted pendulum motion’ of the trunk, the WORM score measures the ‘wobble’ of the upper body as measured from a single-point IMU with a chest-based attachment as shown in FIG. 1. In this illustration, the body frame B, has its x-axis aligned with the initial direction of walk (antero-posterior plane), z-axis aligned with the direction of measured acceleration due to gravity (superio-inferior plane), and y-axis calculated as the cross product of z and x axis (medio-lateral plane).

In another embodiment to calculate the WORM score, first calculate the point pₜ at time step t from the orientation of the body with respect the world frame, ^{W}Rₜ^{B}. The body orientation, ^{W}R^{B}, is obtained from the orientation measured by the single point IMU, ^{W}R̆ₜ^{S}, adjusted by a fixed sensor-to-body rotational offset, ^{B}R₀^{S}, as shown in Equation 15. The sensor-to-body offset, ^{B}R₀^{S}, was calculated by assuming an upright pose (i.e., ^{W}R₀^{B}=I_{3×3}) at t=0 as shown in Equation 16. Finally, point pₜ, which is effectively the x and y coordinates of the body z axis with centre at the origin, is calculated using Equation 17.

$${}^{W} {R}_{t}^{B} = {}^{w} {{R}^{⌣}}_{t}^{S} {( {}^{B} {R}_{0}^{S} )}^{T} \qquad Equation 15$$

$${}^{B} {R}_{0}^{S} = {}^{W} {{R}^{⌣}}_{0}^{S} \qquad Equation 16$$

$${p}_{t} = [ \begin{matrix}1 & 0 & 0 \\ 0 & 1 & 0\end{matrix} ] {}^{W} {R}_{t}^{B} [ \begin{matrix}0 \\ 0 \\ 1\end{matrix} ] \qquad Equation 17$$

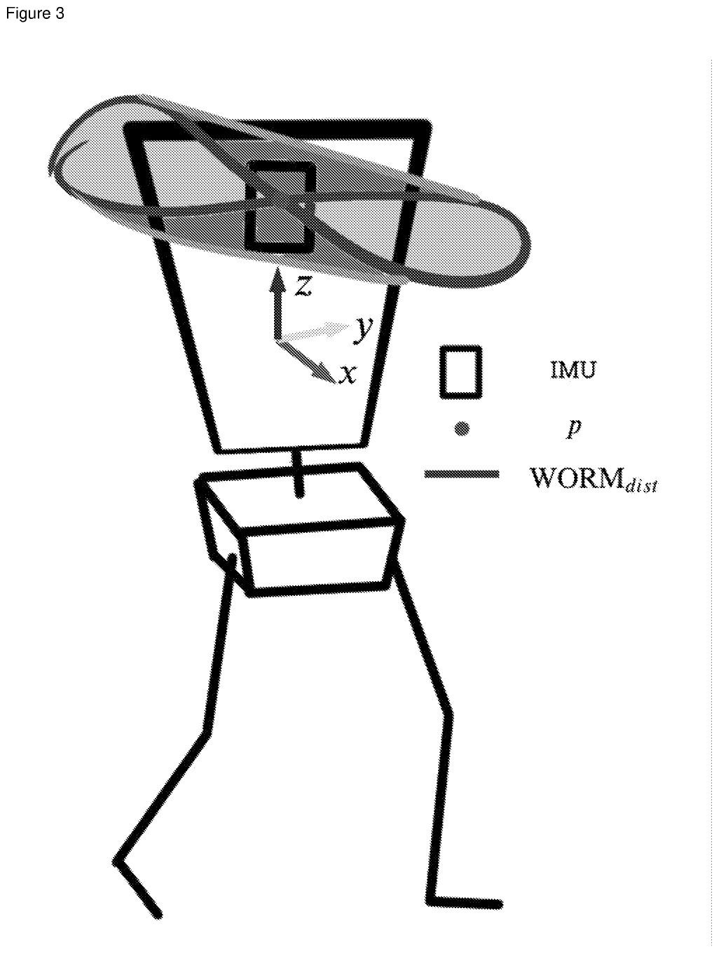

In this embodiment and referring to FIG. 3, the WORM score (or WORMdist) is the distance travelled in the transverse plane travel by pₜ.

In some embodiments the WORM Score is used as a standardised method of assessing walking stability and balance. As described an exemplary embodiment has validated its utility to distinguish fallers from non-fallers within a sample population of 32 participants and demonstrates that the WORM Score identifies an 8-fold increase in fallers compared to non-fallers. Fallers had significant differences in spatiotemporal parameters of gait with lower gait velocity, step length and cadence despite greater step time. Fallers also walked with greater asymmetry (in step time but not step length) and variation (in gait velocity, step length and step time). In terms of falls classification the WORM score provides good discriminative accuracy (AUC>0.90) with gait velocity, step time, step time asymmetry, gait velocity variation.

The study described herein focuses on the application of wearable-sensors for fall risk assessment and through wearable accelerometry the inventors have identified the relevant gait variables with high predictive classification accuracy in distinguishing fallers from non-fallers.

Transverse truncal motion during walking, is independent of (but correlates with) other balance variables. Laboratory-based posturography measurements of centre of pressure (CP), quantify the (weighted average) vertical force vector exerted by a subject on a force platform. These studies have identified anteroposterior (AP) and mediolateral (ML) displacements, velocity and total path lengths of CP as the variables differentiating fallers from non-fallers. However, these CP measurements almost completely ignore the behaviour of the upper half of the body. Moreover, as CP measurements are taken whilst standing unperturbed (static) or whilst moving (dynamic), they assess postural control rather than stability or balance of walking.

Conversely, centre of mass (COM) movement more closely represents balance. Recent, wearable accelerometery studies of walking (dynamic) balance have assessed ambulatory CM movement in fallers as a measure of dynamic balance. These studies typically considered amplitudes and variability of CM motion along (mediolateral or vertical) axial planes, with the sensor-placement most commonly at the approximate centre of mass: along the lumbar vertebrae.

Previous studies on centre of gravity (CG) motion calculated from the ‘mechanical work’ of individual joint segments, established pathological gait patterns to involve greater mechanical energy expenditure. These mechanical inefficiencies likely arise due to compensatory inclinations of the trunk and motions of the upper limbs that seek to offset pathological lower limb biomechanics (and prevent falls). Thus, a greater WORM score (truncal motion) identifies these falls-risk participants with compensatory gait alterations.

The findings of significant differences in a range of gait variables between fallers and non-fallers reinforces the potential use of gait analysis in the detection, preferably early detection, of gait and balance impairments.

The methods described herein provide objective, unsupervised and unobtrusive method of point-of-care testing to assess walking stability and balance in clinical settings and/or home environments. The ‘WORM Score’ provides clinicians, patients, and carers with a quantification of walking instability serving as an accurate, and sensitive biomarker for monitoring functional balance and falls-risk. Further, such identification of gait and balance deficits can prompt timely intervention before a fall occurs and, therefore improve quality of life and avert the need for an additional, or higher-level intervention in the future.

Traditionally, objective monitoring of gait and balance with laboratory-based techniques such as optoelectronic stereophotogrammetry require extensive resources (equipment, trained personnel), and can be time-consuming.

The WORM score is a quantitative measure of walking instability for long-term monitoring and assessment. Accordingly, it can be used in multiple scenarios, including care of falls-prone patients, determining suitability for walking aids, physical therapy, home modifications, altering medication regiments (for geriatric patients) or dose alterations (for instance in Parkinson's disease).

Objective and quantitative falls-risk assessment in the clinical setting via the WORM score also has post-intervention applications in assessing suitability for safe discharge of a patient. Regardless of the intervention (medical or surgical), or presenting complaint (head injury, trauma, or acute/chronic illness) assessment of walking stability prior to discharge benefits patients and health-care systems alike, by minimising post-discharge falls in the community. The WORM score can also inform home care and rehabilitation.

Suitability for falls-preventive interventions (such as walking aids) can also be assessed by the objective and quantitative assessment of walking stability provided by the WORM Score or by a combination of gait metrics. With either age or injury, a point in time may arise when an individual requires mobility assistance. This may include a walking stick, or a walking frame. WORM scores for non-fallers versus fallers are defined herein however the intermediate scores between these 2 points provide an indication that the patient would benefit from walking aid.

In one embodiment, a WORM score for a patient of 0.2 or less is indicative of a minimal risk of falling (i.e this is an example of a minimal fall risk threshold).

A WORM score for a patient of 0.2-0.6 (e.g. a medium fall risk threshold) is indicative of a low risk of falling.

Patients with a minimal or low risk of falling are capable of walking unaided.

A WORM score for a patient of 0.6 (e.g. a medium fall risk threshold)−1.2 (e.g. a high fall risk threshold) is indicative of a medium risk of falling

In one embodiment a WORM score for a patient of more than 1.2 (a high fall risk threshold) is indicative of a high risk of falling.

Advantageously, the WORM score can be calculated from as few at 6 gait cycles. Accordingly, this allows for the systems described herein to include a means to alert the patient, their physician or carer of a falls risk.

In other embodiments two or more of the gait metrics described by equations 1-13, or by any other means known on the art, can be used to determine a fall risk for a subject. In these embodiments the sum of the gait metrics is indicative of fall risk. For example if the sum of the gait metrics meets or exceeds a predetermined threshold value the subject is at risk of falling. In some embodiments if the sum of the gait metrics meets or falls below a predetermined threshold value the subject is at risk of falling.

For example if the sum of gait velocity and step length for a subject is 1.4 or less the subject has a high fall risk. If the sum of gait velocity and step length for a subject is 1.5 or more the subject has a low fall risk.

In another embodiment if the sum of cadence and step time for a subject is 95 or less the subject has a high fall risk. If the sum of gait velocity and step length for a subject is 100 or more the subject has a low fall risk.

In another embodiment if the sum of step time asymmetry and step length asymmetry for a subject is 0.25 or more the subject has a high fall risk. If the sum of step time asymmetry and step length asymmetry 0.2 or less the subject has a low fall risk.

In a further embodiment if the sum of gait velocity variation, step time variation and step length variation for a subject is 0.25 or more the subject has a high fall risk. If the sum of gait velocity variation, step time variation and step length variation is 0.2 or less the subject has a low fall risk.

### EXAMPLES

### Example 1: Analysis of Faller v Non Faller data

### Walking Orientation Randomness Metric (WORM) Score

During walking, the summative motions of individual joint segments accelerates the trunk forwards of the base of support. With each step, the trunk also oscillates laterally along a line of motion situated at the medial borders of either feet. To describe this motion of the trunk, the WORM score measures the ‘wobble’ of the upper body as measured from a single-point IMU with a chest-based attachment as shown in FIG. 3. In this illustration, the body frame B, has its x-axis aligned with the initial direction of walk (antero-posterior plane), z-axis aligned with the direction of measured acceleration due to gravity (superio-inferior plane), and y-axis calculated as the cross product of z and x axis (medio-lateral plane).

To calculate the WORM score, first calculate the point pₜ at time step t from the orientation of the body with respect the world frame, ^{W}Rₜ^{B}. The body orientation, ^{W}R^{B}, is obtained from the orientation measured by the single point IMU, ^{W}R̆ₜ^{S}, adjusted by a fixed sensor-to-body rotational offset, ^{B}R₀^{S}, as shown in equation 15. The sensor-to-body offset, ^{B}R₀^{S}, was calculated by assuming an upright pose (i.e., ^{W}R₀^{B}=I_{3×3}) at t=0 as shown in equation 16 Finally, point pₜ, which is effectively the x and y coordinates of the body z axis with centre at the origin, is calculated using equation 17.

$${}^{W} {R}_{t}^{B} = {}^{W} {{R}^{⌣}}_{t}^{S} {( {}^{B} {R}_{0}^{S} )}^{T} \qquad Equation 15$$

$${}^{B} {R}_{0}^{S} = {}^{W} {{R}^{⌣}}_{0}^{S} \qquad Equation 16$$

$${p}_{t} = [ \begin{matrix}1 & 0 & 0 \\ 0 & 1 & 0\end{matrix} ] {}^{W} {R}_{t}^{B} [ \begin{matrix}0 \\ 0 \\ 1\end{matrix} ] \qquad Equation 17$$

The WORM score (or WORMdist) is the distance travelled in the transverse plane travel by pₜ.

### Study Participants

During their hospital admission, study parameters and risks were discussed, and consent obtained. Inclusion criteria were patients having a primary hospital admission of falls, and the capacity to consent to the study. Exclusion criteria were lacking the ability to walk any distance without a form of support (walking stick or frame). Included patients underwent a semi-structured interview to obtain demographic information and assess eligibility. Participants were asked if they experienced a fall while standing or walking in the previous week and were asked to describe the circumstances of the fall if possible. To be included in the “fallers” group, the fall must have been unrelated to a medication event and the patient must have intact binocular vision without concurrent visual pathologies. Age-matched “non-fallers” were recruited as controls for this study following a similar semi-structured interview.

### Sample Size Calculations

A required sample size of 14 participants per group was calculated using the GPower 3.0 program to achieve at least 80% power given a significant effect size of at least 1. Recruitment target of at least 15 participants was therefore set to account for any potential data losses.

### Procedure

Prior to the walk, participants were fitted at the sternal angle with a inertial measurement unit: the MetaMotion© (MMC) manufactured by Mbientlab Inc. (California, USA). In addition, patients wore a safety belt such that any fall during the subsequent walking event could be prevented by the investigators-3 observers were in close proximity to the patient walking for safety. Following a short initial pause to orient the MMC device, participants walked a self-selected distance (5-50m) along an unobstructed pathway on level ground. Trials were discarded if the patient did (or could) not pause to orient the device, walk more than 5m or required a walking aid during the procedure.

### Wearable Device

The MMC is a wearable sensor which contains a 16 bit 100 Hz triaxial accelerometer for the detection of linear acceleration (anteroposterior, mediolateral, and vertical), a 16 bit 100 Hz triaxial gyroscope for the detection of angular acceleration (pitch, roll and yaw), and a 0.3 μT 25 Hz triaxial magnetometer to assess orientation relative to the Earth's magnetic field (North-South). The data captured by the MMC is stored as a matrix of the values corresponding to each time point (100 captures per second) for up to 20 minutes of walking. For the purposes of this study, the MMC device recorded the entire walking bout, and the data captured was transmitted via Bluetooth™ to an Android™ smartphone running the data processing application developed for this study. The application then uploaded the raw data to a centralised database where data processing was performed to produce the gait metrics for that walking bout.

### Data Processing

The MMC was able to measure dynamic postural sway, data processing was then able to calculate the Walking Orientation Randomness Metric (WORM) score (FIG. 3). WORM is calculated as the distance travelled by point ‘p’ on the upper body (as a centre of gravity) as seen in FIG. 3. The figure-of-eight illustration (FIG. 3) represents the oscillating movement of p through a gait cycle. WORM for the walking bout is calculated as the distance moved by p (the length of the blue outline).

The WORM output from the aforementioned method is then adjusted to varying walking speeds (averaged to time walked) and cadence (averaged to distance walked) to derive the final WORM Score. As such, the numerical WORM output is summed over all the gait cycles for that walking bout, and then divided by the total distance travelled during that walking bout and also divided by the total time taken to complete the walking bout. Thus, the WORM score measures the distance of dynamic postural sway undertaken by the participant's centre of motion, averaged as a mean per metre and per second walked.

### Statistical Analysis

Data analyses were performed using Prism 9 (GraphPad Software). Descriptive statistics were calculated for demographic variables including; age, gender, presence of diabetes and smoking. Spatiotemporal parameters of gait were calculated, and step measurements chosen for calculations of gait asymmetry and variation due to greater reliability compared stride measurements. Differences between fallers and non-fallers were calculated using unpaired two-tailed t-test. Welch's correction was applied for variables with unequal variance and Mann Whitney U test used where non-normal distribution was present. Differences in WORM scores between fallers and non-fallers were calculated using Mann-Whitney U tests. Discriminative ability was assessed using the area under the curve values of receiver operating characteristic curves for each WORM score. Accuracy values were interpreted as follows: 0.5=test due to chance, 0.7-0.9=moderate accuracy, 0.9-1.0=very accurate, 1.0=perfect test. Normality was assessed using Shapiro-Wilk tests and inspection of histograms. Statistical significance was considered with a p-value <0.05.

### Participant Demographics

16 participants recruited as ‘fallers’ had a range of comorbidities including delirium, osteoarthritis, lumbar radiculopathy, scoliosis, vestibular imbalance, cervical myelopathy, foot-drop and stroke-related hemi-paresis. The 16 control participants recruited as ‘non-fallers’ had various comorbidities consistent with their age such as osteoarthritis of the spine and lower extremities, however had no history of falls. Most demographic variables such as age, gender ratios, smoking and diabetic status, body mass indices and height for these participants were not significantly different with the exception of weight (p=0.042) and daily step count (p<0.0001) as seen in Table 1.

TABLE 1
Demographic and clinical characteristics of fallers and non-
fallers. P value represents statistical significance of difference
between groups derived from Unpaired two-tailed t-test (Welch's
correction* applied if unequal variance), Mann Whitney U tests**
(if non-normal distribution) or Fisher's Exact Test^{†}.
Significant findings are bolded

| | Fallers | Non-Fallers | P |
|---|---|---|---|
| N | 16 | 16 | n/a |
| Age (mean ± SD) | 70 ± 17 | 70 ± 9 | 0.6089* |
| Gender ‘M’ (%) | 8 (50) | 10 (62.5) | 0.7224^{†} |
| Height (mean ± SD) | 1.68 ± 0.10 | 1.74 ± 0.11 | 0.1537 |
| Weight (mean ± SD) | 72.99 ± 19.18 | 87.99 ± 20.76 | 0.042 |
| BMI (mean ± SD) | 25.70 ± 5.57 | 29.05 ± 5.59 | 0.0997 |
| Smoking (%) | 1 (6.25) | 0 (0) | n/a |
| Diabetes (%) | 0 (0) | 2 (12.5) | n/a |
| Daily Step Count | 652.0 ± 539.1 | 4419 ± 4053 | <0.0001** |
| (mean + SD) | | | |

### Spatiotemporal Gait Parameters

9 gait characteristics were measured across the four main gait domains of spatial, rhythm/temporal, asymmetry and variation metrics. 8 of these were significantly different between fallers and non-fallers (Table 2), and 7 of these remain significant following Bonferroni's corrections (p=0.05/13=0.0038) for multiple testing.

Faller's have a typical gait pattern of significantly lower gait velocity (p<0.0001), step length (p=0.0016) and cadence (p<0.0001) whilst other parameters are significantly increased including step time (p<0.0001), step time asymmetry (p<0.0001) and variability in terms of gait velocity (p=0.0005), step time (p=0.0002) and step length (p=0.0034). Asymmetry in step length was not found to be significantly different (p=0.0796) in fallers.

The ability of these spatiotemporal parameters of gait to differentiate between fallers and non-fallers was assessed by statistically significant area under the curve (AUC) values of receiver operating characteristic (ROC) curves (Table 3). Good accuracy was found for most gait parameters with the highest accuracy found in gait velocity (AUC=0.9102), step time (AUC =0.9375), asymmetry in step time (AUC=0.9023), and variation in gait velocity (AUC=0.9042).

### WORM Scores

Dynamic instability according to WORM scores (cm) was significantly different (p <0.0001) being 17-fold higher (mean±standard deviation) in fallers (3.644±3.899) compared to non-fallers (0.2108±0.1707). These differences in WORM scores (Tables 4, 5 and 6) show high accuracy (AUC=0.9648) in differentiating fallers from non-fallers with a sensitivity of 87.50% and specificity of 93.75% when selecting a cut-off (WORM>0.5115 cm) with the highest likelihood ratio (14.00).

Faller's have a typical gait pattern of significantly lower gait velocity, step length and cadence whilst other parameters are significantly increased including step time, step time asymmetry and variability in terms of gait velocity, step time and step length.

Dynamic instability according to WORM scores (m/s) was significantly different being 17-fold higher (mean±standard deviation) in fallers compared to non-fallers.

Differences in WORM scores (Tables 4, 5 and 6) show strong accuracy (AUC=0.9648) in differentiating fallers from non-fallers with a sensitivity of 87.50% and specificity of 93.75%

TABLE 2
Gait characteristics of participants (sensor-derived). COV = Coefficient of Variance. P value represents
statistical significance of difference between groups derived from Unpaired two-tailed t-test, Welch's
corrected t-tests* (if unequal variance), or Mann Whitney U tests** (if non-normal distribution).

| | | | Between Group Differences | | |
|---|---|---|---|---|---|
| | | | (Fallers − Non-Fallers) | | |
| | Fallers | Non-Fallers | Mean ± SEM/ | 95% Confidence | |
| | Mean + SD | | Median | Interval | P |
| Spatial Gait Metrics | | | | | |
| Gait Velocity (m/s) | 0.6373 ± 0.2608 | 1.126 ± 0.2518 | −0.4888 ± 0.09064 | −0.6739 to −0.3036 | <0.0001 |
| Step Length (m) | 0.4527 ± 0.1362 | 0.6204 ± 0.1371 | −0.1677 ± 0.04832 | −0.2664 to −0.06904 | 0.0016 |
| Sum | 1.09 | 1.75 | | | |
| Rhythm/Temporal | | | | | |
| Gait Metrics | | | | | |
| Cadence | 84.54 ± 14.26 | 109.4 ± 7.566 | −24.81 ± 4.035 | −33.16 to −16.46 | <0.0001* |
| Step Time (s) | 0.7572 ± 0.1388 | 0.5543 ± 0.03872 | 0.2028 ± 0.03603 | 0.1269 to 0.2787 | <0.0001* |
| Sum | 85.30 | 109.95 | | | |
| Gait Asymmetry | | | | | |
| Step Time | 0.1909 ± 0.1077 | 0.04709 ± 0.02655 | 0.1438 ± 0.02772 | 0.08528 to 0.2024 | <0.0001* |
| Asymmetry (s) | | | | | |
| Step Length | 0.1337 ± 0.08646 | 0.08091 ± 0.03417 | 0.0281 | −0.003040 to 0.07225 | 0.0796* |
| Asymmetry (m) | | | | | |
| Sum | 0.32 | 0.13 | | | |
| Gait Variability | | | | | |
| Gait Velocity | 0.1517 ± 0.07010 | 0.07089 ± 0.02169 | 0.08081 ± 0.01890 | 0.04085 to 0.1208 | 0.0005 |
| Variation (COV) | | | | | |
| Step Time | 0.1885 ± 0.08633 | 0.07533 ± 0.03713 | 0.1132 ± 0.02415 | 0.06260 to 0.1638 | 0.0002 |
| Variation (COV) | | | | | |
| Step Length | 0.2388 + 0.1456 | 0.1047 + 0.04524 | 0.1341 ± 0.03926 | 0.05106 to 0.2171 | 0.0034 |
| Variation (COV) | | | | | |
| Sum | 0.34 | 0.15 | | | |

TABLE 3
AUC values of spatiotemporal gait parameters in
discriminating between Fallers and Non-Fallers.

| | | | 95% Confidence Interval | | |
|---|---|---|---|---|---|
| | | Std. | Upper | Lower | |
| | AUC | Error | Bound | Bound | P |
| Spatial Gait | | | | | |
| Metrics | | | | | |
| Gait Velocity | 0.9102 | 0.05105 | 1.000 | 0.8101 | <0.0001 |
| (m/s) | | | | | |
| Step Length | 0.8008 | 0.07865 | 0.9549 | 0.6466 | 0.0037 |
| (m) | | | | | |
| Rhythm/Temporal | | | | | |
| Gait Metrics | | | | | |
| Step Time (s) | 0.9375 | 0.04539 | 1.000 | 0.8485 | <0.0001 |
| Gait Asymmetry | | | | | |
| Step Time | 0.9023 | 0.05907 | 1.000 | 0.7866 | 0.0001 |
| Asymmetry (s) | | | | | |
| Step Length | 0.6836 | 0.09530 | 0.8704 | 0.4968 | 0.0765 |
| Asymmetry (m) | | | | | |
| Gait Variability | | | | | |
| Gait Velocity | 0.9042 | 0.05644 | 1.000 | 0.7936 | 0.0001 |
| Variation (COV) | | | | | |
| Step Time | 0.8750 | 0.06818 | 1.000 | 0.7414 | 0.0004 |
| Variation (COV) | | | | | |
| Step Length | 0.8250 | 0.07934 | 0.9805 | 0.6695 | 0.0020 |
| Variation (COV) | | | | | |

AUC = Area under the curve.
ROC = Receiver Operating Characteristic.
Std. Error = Standard Error.
COV = Coefficient of variance.

TABLE 4
Differences in WORM Scores (cm) between Fallers and Non-
Fallers. WORM scores calculated as average per metre (mean/m)
and per second (mean/s) of walking. P value represents
statistical significance of Mann Whitney U tests.

| | Fallers | | Non-Fallers | | |
|---|---|---|---|---|---|
| | Mean | SD | Mean | SD | P |
| WORM (cm) | 3.644 | +3.899 | 0.2108 | +0.1707 | <0.0001 |

WORM = Walking Orientation Randomness Metric.
SD = Standard Deviation

TABLE 5
AUC values of ROC curves for WORM (cm) in discriminating
between Fallers and Non-Fallers.

| | | | 95% Confidence Interval | | |
|---|---|---|---|---|---|
| | | Std. | Upper | Lower | |
| | AUC | Error | Bound | Bound | P |
| WORM (cm) | 0.9648 | 0.02757 | 1 | 0.9108 | <0.0001 |

WORM = Walking Orientation Randomness Metric.
AUC = Area under the curve.
ROC = Receiver Operating Characteristic.
Std. Error = Standard Error.

TABLE 6
Diagnostic performance of WORM score in classifying Fallers and Non-Fallers

| Cut-Off | Sensitivity | | Specificity | | PPV | NPV | Likelihood |
|---|---|---|---|---|---|---|---|
| Value | (%) | 95% CI | (%) | 95% CI | (%)* | (%)* | ratio |
| >0.5115 | 87.50 | 63.98% to | 93.75 | 71.67% to | 82.35% | 95.74% | 14.00 |
| (cm) | | 97.78% | | 99.68% | | | |

CI = Confidence Interval.
PPV = Positive predictive value.
NPV = Negative predictive value.
*PPV and NPV are calculated with a prevalence estimate of 0.25 based on findings of literature.

1. A system for determining a fall risk of a subject, the system comprising:
a) an accelerometer configured to output signals indicative of movement of the subject along one or any combination of an x-axis, a y-axis, and a z-axis
b) a magnetometer configured to output signals indicative of variations in position of the subject in a space defined by the x-axis, the y-axis, and the z-axis; and
c) a gyroscope configured to output signals indicative of angular velocity of the subject around one or any combination of the x-axis, the y-axis, and the z-axis;
d) a processor configured to receive the output and analyse the signals to determine for the subject one or any combination of gait metrics:
i. stride time;
ii. stride time variability;
iii. stride cadence;
iv. step time asymmetry;
v. stride length;
vi. stride length variability;
vii. stride length asymmetry;
viii. gait velocity;
ix. gait speed variability; and
x. walking orientation randomness metric (WORM)
wherein the x-axis is a horizontal axis to the ground directed forward of the subject's body; the y-axis being a horizontal axis to the ground directed laterally of the subject's body; and the z-axis is vertical axis to ground.

2. The system of claim 1, wherein the accelerometer, magnetometer and gyroscope are in a sensor unit adapted to be disposed on the subject.

3. The system of claim 1 or 2, wherein the sensor unit further comprises the processor.

4. The system of any one of claims 1 to 3, wherein the system determine the WORM Score by:
i) calculating the point pₜ at time step t from the orientation of the body with respect the world frame, ^{W}Rₜ^{E}.
ii) obtaining the body orientation, ^{W}R^{B}, from the orientation, ^{W}R̆ₜ^{S}, adjusted by a fixed sensor-to-body rotational offset, ^{B}R₀^{S};
wherein the sensor-to-body rotational offset, ^{B}R₀^{S}, is calculated by assuming an upright pose
iii) calculating point pₜ, being the x and y coordinates of the body z axis with centre at the origin; and
wherein the WORM score is the distance travelled in the transverse plane travel by pₜ.

3. The system of claim 2, wherein the body orientation, ^{W}R^{B}, is calculated from the orientation, ^{W}R̆ₜ^{S}, adjusted by the fixed sensor-to-body rotational offset, ^{B}R₀^{S}, according to equation 15:
$${}^{W} {R}_{t}^{B} = {}^{W} {{R}^{⌣}}_{t}^{S} {( {}^{B} {R}_{0}^{S} )}^{T} \qquad Equation 15$$

4. The system of claim 2 or 3, wherein the sensor-to-body rotational offset, ^{B}R₀^{S}, is calculated by assuming an upright pose (^{W}R₀^{B}=I_{3×3}) at t=0 according to equation 16:
$${}^{B} {R}_{0}^{S} = {}^{W} {{R}^{⌣}}_{0}^{S} \qquad Equation 16$$

5. The system of any one of claims 2 to 4, wherein point pₜ, is calculated using equation 17:
$${p}_{t} = [ \begin{matrix}1 & 0 & 0 \\ 0 & 1 & 0\end{matrix} ] {}^{W} {R}_{t}^{B} [ \begin{matrix}0 \\ 0 \\ 1\end{matrix} ] \qquad Equation 17$$

6. The system of any one of claims 1 to 3, wherein the WORM Score is calculated using Equation 14:
$$WORM = \frac{{\sum }_{i = 1}^{n} Li}{n} \qquad Equation 14$$
where Li is total length of the path taken by a point X relative to a point O in the horizontal plane during a meter i of a walking bout of n meters.

7. The system of any one of claims 1 to 3, wherein the processor is configured to determine a gait stability score from any two, three, four, five, six, seven, eight, or nine of gait metrics i-ix.

8. The system of claim 7 wherein determining the gait stability score is determined by summing:
gait velocity and step length;
cadence and step time;
step time asymmetry and step length asymmetry; or
gait velocity variation, step time variation and step length variation.

9. A method of determining a fall risk of a subject comprising determining a gait stability score for the subject from at least two of
i. stride time;
ii. stride time variability;
iii. stride cadence;
iv. step time asymmetry;
v. stride length;
vi. stride length variability;
vii. stride length asymmetry;
viii. gait velocity;
ix. gait speed variability; or
wherein fall risk is determined by calculating a walking orientation randomness metric (WORM) score wherein one or both of the gait stability score or WORM score is indicative of a fall risk.

10. The method of claim 9, wherein WORM Score is calculated by:
i) calculating the point pₜ at time step t from the orientation of the body with respect the world frame, ^{W}Rₜ^{B}.
ii) obtaining the body orientation, ^{W}R^{B}, from the orientation, ^{W}R̆Rₜ^{S}, adjusted by a fixed sensor-to-body rotational offset, ^{B}R₀^{S};
wherein the sensor-to-body rotational offset, ^{B}R₀^{S}, is calculated by assuming an upright pose
iii) calculating point pₜ, being the x and y coordinates of the body z axis with centre at the origin; and
wherein the WORM score is the distance travelled in the transverse plane travel by pₜ.

11. The method of claim 10, wherein the body orientation, ^{W}R^{B}, is calculated from the orientation, ^{W}R̆ₜ^{S}, adjusted by the fixed sensor-to-body rotational offset, ^{B}R₀^{S}, according to equation 15:
$${}^{W} {R}_{t}^{B} = {}^{W} {{R}^{⌣}}_{t}^{S} {( {}^{B} {R}_{0}^{S} )}^{T} \qquad Equation 15$$

12. The method of claim 10 or 11, wherein the sensor-to-body rotational offset, ^{B}R₀^{S}, is calculated by assuming an upright pose (^{W}R₀^{B}=I_{3×3}) at t=0 according to equation 16:
$${}^{B} {R}_{0}^{S} = {}^{W} {{R}^{⌣}}_{0}^{S} \qquad Equation 16$$

13. The system of any one of claims 10 to 12, wherein point pₜ, is calculated using equation 17:
$${p}_{t} = [ \begin{matrix}1 & 0 & 0 \\ 0 & 1 & 0\end{matrix} ] {}^{W} {R}_{t}^{B} [ \begin{matrix}0 \\ 0 \\ 1\end{matrix} ] \qquad Equation 17$$

14. The method of claim 9 wherein the WORM Score is calculated using equation 14:
$$WORM = \frac{{\sum }_{i = 1}^{n} Li}{n} \qquad Equation 14$$
where Li is total length of the path taken by a point X relative to a point O in the horizontal plane during a meter i of a walking bout of n meters.

15. The method of any one of claims 9 to 14, wherein a WORM score of above a predetermined threshold indicative of a high fall risk (high fall risk threshold).

16. The method of claim 15, wherein the high fall risk threshold is 1.2.

17. The method of any one of claims 9 to 14, wherein a WORM score below a predetermined threshold indicative of a minimal risk of falling (minimal fall risk threshold).

18. The method of claim 17 wherein the minimal fall risk threshold is 0.2.

19. The method of any one of claims 9 to 14, wherein a WORM score between a minimal fall risk threshold and a high fall risk threshold is indicative of a medium risk of falling.

20. The method of claim 19, wherein the minimal fall risk threshold is 0.2 and the high fall risk threshold is 1.2.

21. The method of any one of claims 9 to 14, wherein a WORM score of between a medium fall risk threshold and a high fall risk threshold is indicative of a medium risk of falling

22. The method of claim 21, wherein the medium fall risk threshold is 0.6 and the high fall risk threshold is 1.2.

23. The method of claim 8 or 9 wherein the gait stability score is the sum of gait velocity and step length a gait stability score of 1.4 or less is indicative of a fall risk.

24. The method of any one of claim 8 or 9, wherein the gait stability score is the sum of cadence and step time a gait stability score of 95 or less is indicative of a fall risk.

25. The method of any one of claim 8 or 9, wherein the gait stability score is the sum of step time asymmetry and step length asymmetry a gait stability score of 0.25 or more is indicative of a fall risk.

26. The method of any one of claim 8 or 9, wherein the gait stability score is the sum of gait velocity variation, step time variation and step length variation and wherein a gait stability score of 0.25 or more is indicative of a fall risk.
